(19) United States
(12) Patent Application Publication
(10) Pub. No.: US 2005/0203540 A1
(43) Pub. Date: Sep. 15, 2005

(54) PELVIS LEVEL

(76) Inventor: Joseph E. Broyles, Baton Rouge, LA (US)

Correspondence Address: REGINALD F ROBERTS JR, PO BOX 4535, BATON ROUGE, LA, 70821-4535, US

(21) Appl. No.: 10/708,521

(22) Filed: Mar. 9, 2004

(51) Int. Cl. A61B 17/58

(52) U.S. Cl. 606/102

(57) ABSTRACT

Instrumentation and method for determining the orientation of the pelvic bone during hip-replacement surgery. Instrumentation includes a pelvis frame and a pelvis level. The pelvis frame is used for performing an operation which provides preliminary information for determining the position of the pelvic bone, and includes pads which are specifically contoured for contact with the pelvic bone. The pelvis level is used in combination with the pelvis frame to determine the exact position of the patient's pelvic bone, and includes a ball level which is capable of withstanding steam sterilization.

### BACKGROUND OF INVENTION

The present invention relates to surgery. More specifically, the invention relates to hip-replacement surgery. Such surgery is frequently performed as a treatment for arthritis of the hip. Sometimes, the hip ball can, after the operation, slip out of the hip socket. Such an occurrence is technically and medically referred to as a dislocation. A common cause of such dislocation is improper positioning of the acetabular component (hip socket) in the patient's pelvic bone. There are generally accepted angles for placement of the acetabular component that an orthopedic surgeon attempts to attain while performing the surgery. To do so, the surgeon must know or determine the orientation of the patient's pelvic bone. Herein lies the problem. The patient is covered with sterile surgical drapes, and only a small portion of the bone is visible. The patient may be leaning forward or backward, and there is no accurate method to assess his or her exact position or orientation. The present invention provides a solution to this problem.

### SUMMARY OF INVENTION

In general, the present invention provides instrumentation and method for determining the orientation of the pelvic bone during hip-replacement surgery.

The instrumentation comprises a pelvis frame and a pelvis level.

The pelvis frame comprises (a) a first rigid elongated member; (b) a second rigid elongated member mounted on the first rigid member in a perpendicular relationship thereto; (c) first and second pads attached to the first rigid elongated member in a perpendicular configuration; (d) a third pad attached to the second rigid elongated member in a perpendicular configuration; and (e) means for varying the position of the first, second, and third pads, and for fixating said position as required, for effecting orientation-determining contact of the first, second, and third pads with the pelvic bone of a patient undergoing hip-replacement surgery. The first, second, and third pads are contoured to conform to portions of the pelvic bone which said pads contact.

The pelvis level comprises a housing which includes first and second parallel straight-line openings extending therethrough, for insertion therein of first and second wires used to effect temporary connection of the housing to the pelvic bone of a patient undergoing hip-replacement surgery. The pelvis level further comprises a level, disposed in the housing under a transparent cover, for determination of a level position of the housing.

The method comprises (a) providing a pelvis frame comprising a first rigid elongated member; a second rigid elongated member mounted perpendicularly on the first rigid elongated member; first and second pads attached perpendicularly to the first rigid elongated member; a third pad attached perpendicularly to the second rigid elongated member; and means for varying the position of the first, second, and third pads, and for fixating said position as required, for effecting orientation-determining contact of the first, second, and third pads with a patient undergoing hip-replacement surgery; (b) providing a pelvis level including a level disposed in a housing which includes first and second parallel straight-line openings extending therethrough; (c) adjusting the pelvis frame so that the first and second pads rest on the anterior superior iliac spines of the patient undergoing the hip-replacement surgery; (d) adjusting the pelvis frame so that the third pad rests on the pubic symphysis of the pelvic bone of the patient undergoing the surgery; (e) drilling first and second wires into the anterior superior iliac spine through first and second openings in the first or second pad contacting the anterior superior iliac spine on the side on which the surgery is to be performed; (f) removing the pelvis frame from contact with the patient; (g) turning the patient from back contact to side contact with an operating-room bed; (h) beginning the hip-replacement surgery; (i) at a point during the surgery that the acetabular component of the pelvic bone is to be replaced, sliding the pelvis level over the first and second wires through the first and second holes in the pelvis level; (j) adjusting position of the operating-room bed as required until the pelvis level indicates level position; and (k) replacing the acetabular component of the pelvic bone.

### BRIEF DESCRIPTION OF DRAWINGS

FIG. 1A is a plan view of a pelvis frame for hip-replacement surgery, made in accordance with the principles of the present invention.

FIG. 1B is a front elevation of the pelvis frame shown in FIG. 1A.

FIG. 1C is a side elevation of the pelvis frame shown in FIG. 1A.

FIG. 2 is a schematic representation of a portion of the body of a patient undergoing hip-replacement surgery, lying on his/her back, with the pelvis frame of FIGS. 1A-1C resting thereon.

FIG. 3A is an isometric view of a bubble level for hip-replacement surgery, made in accordance with the principles of the present invention.

FIG. 3B is a plan view of the bubble level shown in FIG. 3A.

FIG. 3C is a side view of the bubble level shown in FIGS. 3A and 3B.

FIG. 4A is an isometric view of a ball level for hip-replacement surgery, made in accordance with the principles of the present invention.

FIG. 4B is a plan view of the ball level shown in FIG. 4A.

FIG. 4C is a side view of the ball level shown in FIGS. 4A and 4B.

FIG. 5A is a schematic representation of the side of the body of a patient lying on his/her side, with two wires in the pelvis, and the bubble or ball level inserted over the wires.

FIG. 5B is a schematic representation of the front of the body of a patient lying on his/her side, with two wires in the pelvis, and the bubble or ball level inserted over the wires.

### DETAILED DESCRIPTION

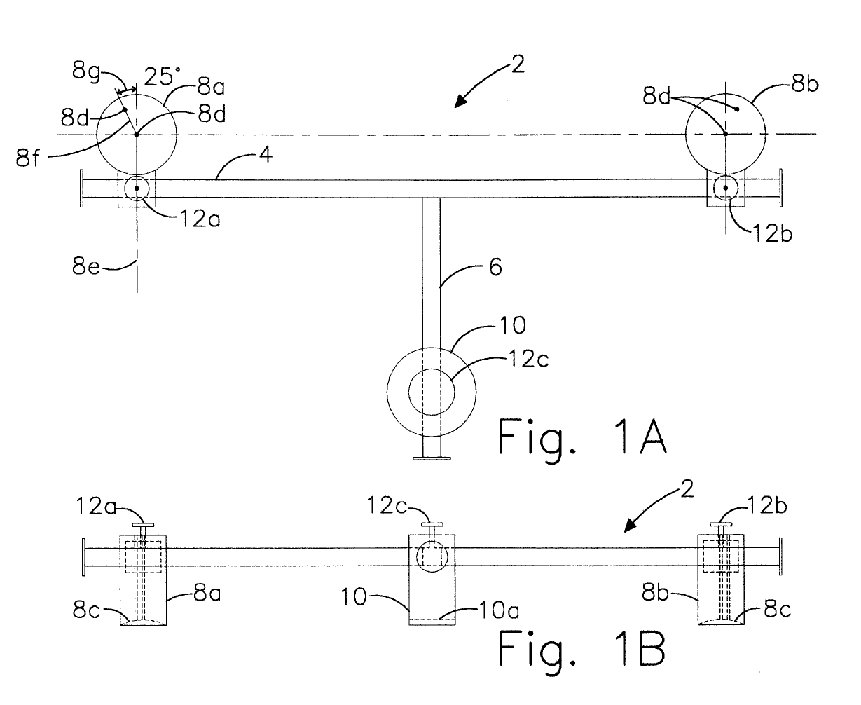

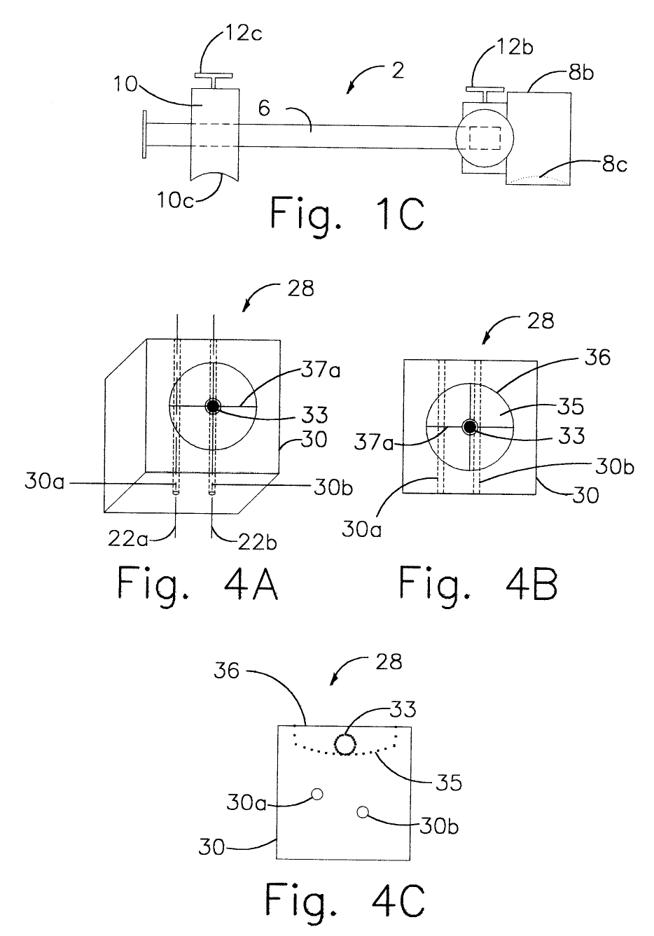

More specifically, reference is made to FIGS. 1A-1C, in which is shown a pelvis frame for hip-replacement surgery, made in accordance with the principles of the present invention, and generally designated by the numeral 2.

The pelvis frame 2 comprises a first rigid elongated member 4; a second rigid elongated member 6 mounted on the first rigid elongated member 4 in a perpendicular relationship thereto; first and second pads 8a and 8b attached to the first rigid elongated member 4 in a perpendicular configuration; a third pad 10 attached to the second rigid elongated member 6 in a perpendicular configuration; and screw-down knobs 12a, 12b, and 12c. The screw-down knobs 12a, 12b, and 12cprovide the means for varying the position of the first, second, and third pads 8a, 8b, and 10, and for fixating said position as required, for effecting orientation-determining contact of the first, second, and third pads 8a, 8b, and 10 with the pelvic bone of a patient undergoing hip-replacement surgery.

As seen best in FIG. 1B and FIG. 1C, the first, second, and third pads 8a, 8b, and 10 are contoured to conform to portions of the pelvic bone which said pads contact. More specifically, pads 8a and 8b are cylindrical, with a contact surface 8c which is concave. By concave is meant that the surface 8c is concave as viewed in FIG. 1B. The third pad 10 is cylindrical, with a contact surface 10a which is saddle-shaped. By saddle-shaped is meant that the surface 10a is saddle-shaped as viewed in FIG. 1C. The first and second pads 8a and 8b include guide openings 8d for insertion of wires used to determine the orientation of the patient undergoing surgery. The width of the guide openings 8d is preferably about two millimeters. Disposition of the guide openings 8d is defined in FIG. 1A. Intersection of a line 8e drawn perpendicular to the first rigid elongated member 4 with a line 8f drawn between and connecting the guide openings 8d to one another defines an angle 8g of from about twenty degrees to about thirty degrees. Preferably, the angle 8g has a magnitude of from about twenty-four degrees to about twenty-six degrees. Most preferably, the magnitude of the angle 8g is about twenty-five degrees.

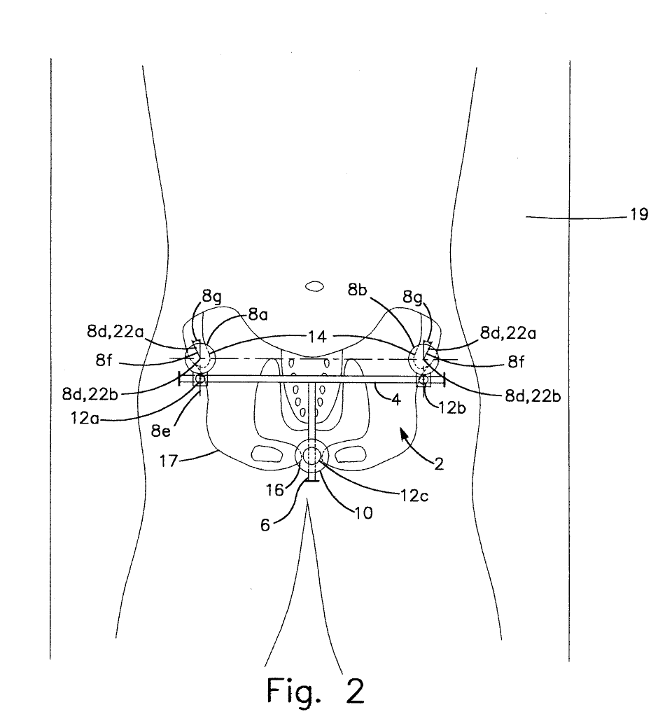

Reference is now made to FIG. 2, in which is represented a portion of the anatomy of a patient undergoing hip-replacement surgery, with the pelvis frame 2 resting on the patient's abdomen. More specifically, the first and second pads 8a and 8b contact the patient's anterior superior iliac spines 14, while the third pad 10 rests on the pubic symphysis 16 of the patient's pelvic bone 17.

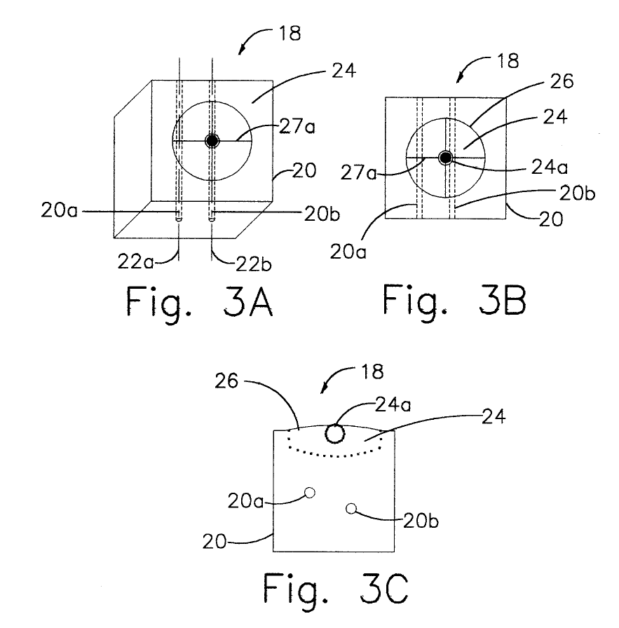

Reference is now made to FIGS. 3A-3C, in which is shown a bubble level for hip-replacement surgery, made in accordance with the principles of the present invention, and generally designated by the numeral 18. The bubble level 18 comprises a housing 20 which includes first and second parallel straight-line openings 20a and 20b extending therethrough, for insertion therein of first and second wires 22a and 22b. The wires 22a and 22b are used to effect temporary connection of the housing 20 to the pelvic bone 17 of the patient undergoing the hip-replacement surgery. The bubble level 18 further comprises a liquid 24 including a bubble 24a, disposed under a convex transparent cover 26. By convex is meant convex as viewed in FIGS. 3A-3C.

Reference is now made to FIGS. 4A-4C, in which is shown a ball level for hip-replacement surgery, made in accordance with the principles of the present invention, and generally designated by the numeral 28. The ball level 28 comprises a housing 30 which includes first and second parallel straight-line openings 30a and 30b extending therethrough, for insertion therein of the first and second wires 22a and 22b. As for the bubble level 18, the wires 22a and 22b are used for the temporary connection of the housing 30 to the pelvic bone 17 of the patient undergoing the surgery. The ball level 28 further comprises a ball 33 disposed on a concave surface 35 between a transparent cover 36 and the concave surface 35. By concave is meant concave as viewed in FIGS. 4A-4C. The ball level 28 is beneficially made of materials that can withstand steam sterilization. Thus, the ball 33 and the housing 30 are preferably made of metal, polytetrafluoroethylene, a heat-resistant plastic, a ceramic, or other heat-resistant material. The transparent cover 36 is preferably made of Pyrex® glass or any other transparent heat-resistant material.

For a description of the surgical method of the present invention, reference is again made to FIGS. 2-5B.

As shown in FIG. 2, after anesthesia has been administered to the patient, the pelvis frame 2 is placed on the front of the patient's lower abdominal area, with the patient lying on his or her back on an operating room bed 19. The pelvis frame 2 is adjusted so that the first and second pads 8a and 8b rest on the anterior superior iliac spines 14, and the third pad 10 rests on the public symphysis 16 of the patient's pelvic bone 17. On the side that the hip replacement is to be performed, the two wires 22a and 22b are drilled into the anterior superior iliac spine 14 through the openings 8d in the first or second pad 8a or 8b. The pelvis frame 2 is then removed, leaving the wires 22a and 22b in the pelvis.

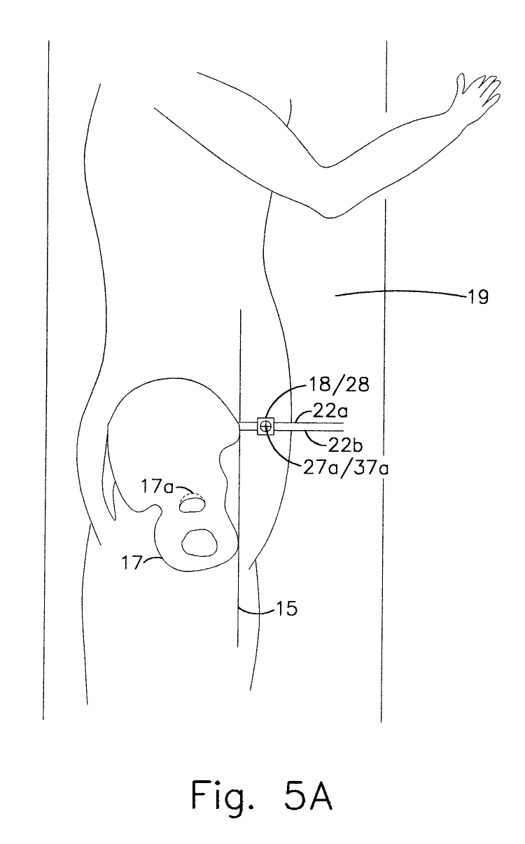

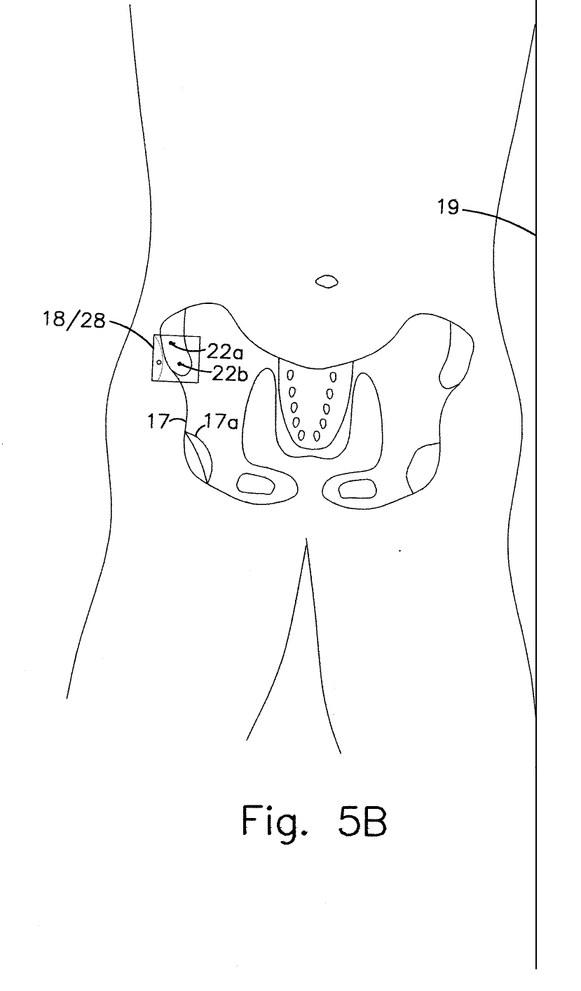

As shown in FIG. 5A and 5B, the patient is then turned side-ways on the operating room bed 19, and the hip surgery is begun. FIG. 5A depicts a patient having a right hip surgery. The wires 22a and 22b are perpendicular to the coronal plane 15 of the pelvis. At the point during the surgery that the acetabular component 17a of the pelvic bone 17 is to be replaced, the pelvis level 18 (FIGS. 3A-3C) or 28 (FIGS. 4A-4C) is slid over the wires 22a and 22b through the openings 20a and 20b (FIGS. 3A-3C) or 30a and 30b (FIGS. 4A-4C) in the housing 20 (FIGS. 3A-3C) or 30 (FIGS. 4A-4C). The operating room bed 19 is then adjusted with its built-in controls until the bubble 24a (FIGS. 3A-3C) or the ball 33 (FIGS. 4A-4C) is centered, indicating that the pelvis is perpendicular to the floor of the operating room, and providing the surgeon with the necessary information to insert the acetabular component 17a at the correct angle. Crosshairs 27a (FIGS. 3A and 3B) or 37a (FIGS. 4A and 4B) on the pelvis level 18 (FIGS. 3A-3C) or 28 (FIGS. 4A-4C) are parallel to the coronal plane 15 of the pelvis as depicted in FIG. 5A. The crosshairs 27a (FIGS. 3A and 3B) and 37a (FIGS. 4A and 4B) therefore provide additional information about pelvic position which is not accounted for by centering the bubble or the ball. At the end of the operation, the alignment wires 22a and 22b are removed from the patient's pelvis.

While certain embodiments and details have been described to illustrate the present invention, it will be apparent to those skilled in the art that many modifications are possible and can be made without departing from the spirit, basic principles, and scope of the invention.

1. A pelvis frame for hip-replacement surgery on a patient, the pelvis frame comprising:
a. a first rigid elongated member;
b. a second rigid elongated member mounted on the first rigid elongated member in a perpendicular relationship thereto;
c. first and second pads attached to the first rigid elongated member in a perpendicular configuration;
d. a third pad attached to the second elongated member in a perpendicular configuration; and
e. means for varying position of the first, second, and third pads, and for fixating said position as required, for effecting orientation-determining contact of the first, second, and third pads with pelvic bone of the patient; the first, second, and third pads being contoured to conform to portions of the pelvic bone which said pads contact.

2. The pelvis frame of claim 1, wherein the first and second pads include openings for insertion therethrough of first and second wires used to determine the patient's orientation.

3. A pelvis level for hip-replacement surgery on a patient, the pelvis level comprising:
a. a housing which includes first and second parallel straight-line openings extending therethrough, for insertion therein of first and second wires used to effect temporary connection of the housing to pelvic bone of the patient; and
b. a level, disposed in the housing under a transparent cover, for determination of a level position of the housing.

4. The pelvis level of claim 3, wherein the pelvis level is a bubble level comprising a liquid including a bubble, disposed under a convex transparent cover.

5. The pelvis level of claim 3, wherein the pelvis level is a ball level comprising a ball disposed on a concave surface between the transparent cover and the concave surface.

6. The pelvis level of claim 5, wherein the ball, the cover, and the housing are made of a material capable of with-standing steam sterilization.

7. A method for determining orientation of pelvic bone of a patient undergoing hip-replacement surgery, the method comprising the steps of:
a. providing a pelvis frame comprising a first rigid elongated member; a second rigid elongated member mounted perpendicularly on the first rigid elongated member; first and second pads attached perpendicularly to the first rigid elongated member; a third pad attached perpendicularly to the second rigid elongated member; and means for varying position of the first, second and third pads, and for fixating said position as required, for effecting orientation-determining contact of the first, second, and third pads with the patient undergoing the surgery;
b. providing a pelvis level including a level disposed in a housing which includes first and second parallel straight-line openings extending therethrough;
c. adjusting the pelvis frame so that the first and second pads rest on the anterior superior iliac spines of the patient undergoing the surgery;
d. adjusting the pelvis frame so that the third pad rests on the pubic symphysis of the pelvic bone of the patient undergoing the surgery;
e. drilling first and second wires into the anterior superior iliac spines through first and second openings in the first or second pad contacting the anterior superior iliac spine on the side on which the surgery is to be performed;
f. removing the pelvis frame from contact with the patient;
g. turning the patient from back contact to side contact with an operating-room bed;
h. beginning the hip-replacement surgery;
i. at a point during the surgery that acetabular component in the pelvic bone is to be replaced, sliding the pelvis level over the first and second wires through the first and second openings in the first or second pad on the side on which the surgery is being performed;
j. adjusting position of the operating-room bed as required until the pelvis level indicates level position; and
k. replacing the acetabular component in the pelvic bone.

8. The method of claim 7, wherein the pelvis level is a bubble level comprising a liquid including a bubble, disposed under a convex transparent cover.

9. The method of claim 7, wherein the pelvis level is a ball level comprising a ball disposed on a concave surface between the concave surface and a transparent cover.

10. The method of claim 9, wherein the ball, the cover, and the housing are made of a material capable of with-standing steam sterilization.
